(19) United States
(12) Patent Application Publication
(10) Pub. No.: US 2010/0286030 A1
(43) Pub. Date: Nov. 11, 2010

(54) NOVEL BACTERIOCIN FROM A NEW STREPTOMYCES SPECIES

(75) Inventors: M. Heath Farris, Vienna, VA (US); Perry F. Churchill, Tuscaloosa, AL (US); Julie B. Olson, Coker, AL (US)

Correspondence Address: McKeon Meunier Carlin & Curfman LLC, 817 W. Peachtree Street, Suite 900, Atlanta, GA, 30308, US

(73) Assignee: THE UNIVERSITY OF ALABAMA, Tuscaloosa, AL (US)

(21) Appl. No.: 12/681,255

(22) PCT Filed: May 16, 2008
(86) PCT No.: PCT/US2008/006279

§ 371 Date: Jul. 8, 2010

Related U.S. Application Data
(60) Provisional application No. 60/997,883, filed on Oct. 5, 2007.

(51) Int. Cl. A01N 63/02; C12N 1/20; C07K 7/08; C07H 21/00; A01P 1/00

(52) U.S. Cl. 514/2.8; 435/253.5; 530/326; 536/23.7; 514/2.4

(57) ABSTRACT

Provided is an isolated novel Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria. Also provided are novel purified polypeptides, and fragments thereof, and isolated nucleic acids from the bacterium. Further provided are methods of using the bacterium and the purified polypeptides, and fragments thereof, to preserve food and prevent bacterial contamination of food.

### CROSS-REFERENCE TO RELATED APPLICATIONS

This application claims the benefit of U.S. Provisional Application No. 60/997,883, filed Oct. 5, 2007, which is hereby incorporated herein by reference in its entirety.

### ACKNOWLEDGEMENTS

This invention was made with government support under Grant 1 R15 GM 069402-01 awarded by the National Institutes of Health. The government has certain rights in the invention.

### BACKGROUND OF THE INVENTION

1. Field of the Invention

This invention relates generally to the isolation of a novel Gram-positive non-motile, non-spore-forming, aerobic bacterium designated Streptomyces scopuloiridis, found in a woodland bluff of Lynn, Ala., that produces a broad-spectrum bacteriocin that kills or inhibits the growth of Gram-positive or Gram-negative target bacteria, and uses thereof.

2. Background Art

Food processing and preservation are still associated with economic losses due to a number of factors including a limited shelf life, food spoilage, and the transmission of food-borne pathogens.

Studies have shown that bacteriocins, ribosomally synthesized proteins or peptides produced by bacteria that kill or inhibit the growth of microorganisms [1], can offer several benefits. These benefits include extending the shelf life of foods, decreasing spoilage, and reducing the risk of exposure to food-borne pathogens. Additionally, bacteriocins have a novel mode of action, typically acting on the cell membrane, so that there is no cross-resistance with approved and marketed antibiotics. Bacteriocins can therefore be used in combination with approved and marketed antibiotics to make it less likely for resistant strains to develop. Moreover, bacteriocins target prokaryotes but not eukaryotes, making them safe for human consumption. Bacteriocins can also reduce the use of chemical preservatives, as well as facilitate the marketing of foods that are less acidic, have a lower salt content, or have a higher water content than foods now available.

Most bacteriocins are cationic and amphiphilic, which allow them to target and permeabilize the bacterial cell membrane as the preferred mode of killing [2]. All major lineages of Bacteria produce bacteriocins, and unique bacteriocins can be found within a given bacterial species [3, 4]. The bacteriocins and bacteriocin-like inhibitory substances (BLIS) of Gram-positive bacteria are currently divided into four broad groups: (I) lantibiotics (containing lanthionine, 3-methyllanthionine, dehydroalanine, and/or dehydrobutyrine), (II) small non-modified peptides (<10 kDa), (III) large proteins (>10 kDa), and (IV) cyclic peptides [5].

The bacteriocins of the lactic acid bacteria (LAB) group are the most well-defined of the Gram-positive bacteriocins. Because LAB are generally regarded as safe for human consumption, their bacteriocins, for example nisin, have received attention as natural food preservatives [6, 7, 8, 9]. Bacteriocins of Gram-positive bacteria not related to the LAB have also shown utility as prophylactics against organisms that would contaminate foodstuffs and cause disease in humans [10, 11]. Beyond the food industry, bacteriocins of Gram-positive bacteria have potential for use as human medical antibiotics [12, 13, 14, 15, 16, 17], animal veterinary antibiotics [18, 19], and agricultural biocontrol agents [20, 21]. While most of the bacteriocins of Gram-positive bacteria are described as small, heat-stable cationic peptides, several have been shown to be high-molecular-weight, heat-labile BLIS [22, 23, 24, 25, 26].

To date, only one such bacteriocin, nisin, has been approved for use by the United States Food and Drug Administration (FDA) as an antimicrobial agent for use on casings for frankfurters and on cooked meat and poultry products. Because different bacteriocins have different spectra of bacteriocidal activity, and because nisin-resistant L. monocytogenes strains have been reported [27], what is needed in the art is a safe, new broad-spectrum bacteriocin, for example, from a Streptomyces species. Streptomyces sp. strain RB72, a novel bacterium, isolated from a woodland bluff of Lynn, Ala., produces a 27 kDa, heat-labile bacteriocin-like enzyme that is referred herein as BLIS RB72. This is the first characterization of a bacteriocin-like enzyme from a Streptomyces bacterium with broad-spectrum activity directed against both Gram-positive and Gram-negative target bacteria. This bacteriocin has a variety of industrial uses, for example, the preservation of food or the prevention of bacterial contamination of food.

### SUMMARY OF THE INVENTION

In accordance with the purposes of this invention, as embodied and broadly described herein, this invention, in one aspect, relates to an isolated Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria.

In another aspect, provided is an isolated polypeptide comprising a 15 amino acid sequence identified as SEQ ID NO:2, wherein the polypeptide has a weight of about 27 kDa as visualized by SDS-PAGE.

In another aspect, provided is an isolated nucleic acid comprising a nucleotide sequence identified as SEQ ID NO:3, wherein the nucleic acid encodes a polypeptide comprising a 15 amino acid sequence identified as SEQ ID NO:2, wherein the polypeptide has a weight of about 27 kDa as visualized by SDS-PAGE.

In yet another aspect, provided is a method of killing or inhibiting the growth of a target bacterium, comprising contacting the target bacterium with an effective amount of a bacteriocin comprising a polypeptide comprising a 15 amino acid sequence identified as SEQ ID NO:2, wherein the polypeptide has a weight of about 27 kDa as visualized by SDS-PAGE, wherein the polypeptide, and/or fragments thereof, kill or inhibit the growth of Gram-positive or Gram-negative target bacteria, whereby contacting the target bacterium with the bacteriocin kills or inhibits the growth of the target bacterium.

In another aspect, provided is a method of growing a Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria, comprising culturing the bacterium at a temperature and in a medium effective to promote growth of the bacterium.

In yet another aspect, provided is a method of preserving food or preventing bacterial contamination of food, comprising contacting the food with an effective amount of a bacteriocin comprising a polypeptide comprising a 15 amino acid sequence identified as SEQ ID NO:2, wherein the polypeptide has a weight of about 27 kDa as visualized by SDS-PAGE, wherein the polypeptide, and/or fragments thereof, kill or inhibit the growth of Gram-positive or Gram-negative target bacteria, whereby contacting the food with the bacteriocin preserves the food or prevents bacterial contamination of the food.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate one embodiment of the invention and together with the description, serve to explain the principles of the invention.

FIG. 1 shows the ammonium sulfate precipitation standard curve of BLIS RB72. Lytic activity recovered from ultrafiltration-concentrated Streptomyces sp. strain RB72 supernatant with increasing concentrations of ammonium sulfate as monitored by dye-release assays of Remazol brilliant blue R-labeled S. coelicolor cells is shown. The highlighted portion of the plot indicates the percent saturation range that precipitated the most lytic activity.

FIG. 2 shows the titration of BLIS RB72 using DEAE Sepharose® anion exchange chromatography. The line graph denotes the protein concentrations as measured by absorbance at 280 nm within each 35 mL fraction collected from a DEAE Sepharose® anion exchange column (pH 8.2) containing bound proteins from ammonium sulfate precipitated supernatant proteins of Streptomyces sp. strain RB72. Peaks A, B, C, and D correspond to the protein recovered after increasing the ionic strength of the eluant solution from 0 mM to 100 mM (A), 250 mM (B), 500 mM (C), and 2 M (D). The shaded bars reflect the amount of lytic activity contained within each peak as measured by a quantitative dye-release assay with Remazol brilliant blue R-labeled S. coelicolor substrate.

FIGS. 3A-3C show the preparation of BLIS RB72 using size exclusion FPLC. (A) Elution profile (1 ml/min flow rate) of ammonium sulfate precipitated, DEAE anion exchange chromatography purified supernatant of Streptomyces sp. strain RB72. The highlighted portion of the profile corresponds to the collected fraction (16.60-18.11 min) that produced lytic activity in dye-release assays. (B) Elution profile (0.5 ml/min flow rate) of the collected fraction from the first round of gel filtration. The highlighted portion of the profile corresponds to the collected fraction (34.00-35.65 min) that produced lytic activity in dye-release assays. (C) Elution profile (0.5 ml/min flow rate) of the purified BLIS RB72 collected after the second round of FPLC.

FIGS. 4A-4C show the preparation of Streptomyces sp. strain RB72 bacteriocin BLIS RB72. (A) Stepwise purification of the bacteriocin as shown on SDS-PAGE. Lanes labeled L contain molecular mass standards. Lane 1, ultrafiltration-concentrated Streptomyces sp. strain RB72 72-h supernatant; lane 2, ammonium sulfate precipitation fraction; lane 3, 2M DEAE anion-exchange chromatography fraction; lane 4, FPLC size exclusion chromatography fraction, 7 μg preparation. (B) Elution profile of purified bacteriocin using FPLC size exclusion chromatography monitored by absorbance at 280 nm. (C) Bacteriolytic activity of the purified enzyme as viewed by an SDS-PAGE zymograph containing heat-killed S. coelicolor substrate. After renaturation, incubation, and staining, a band of bacteriolytic activity appeared as a clear zone in a dark background as indicated by the arrow.

FIG. 5 shows the analysis of purity of BLIS RB72 during and after size exclusion-FPLC preparation using 20% polyacrylamide SDS-PAGE. Lane L, molecular mass standards; lane A, purified BLIS RB72 (7 μg) after two rounds of SE-FPLC; lane B, BLIS RB72-containing preparation after one round of SE-FPLC.

FIG. 6 shows the effect of bacteriolytic enzyme on the viability of the Bacillus subtilis 168 indicator culture. Symbols: Δ, control (no enzyme, addition of buffer only); ▪, addition of bacteriolytic enzyme. R², regression coefficient for the best-fit third-order polynomial.

FIG. 7 shows a neighbor-joining phylogenetic tree based on near-complete 16S rRNA gene sequences showing the relationship between strain RB72 and 23 Streptomyces species. Numbers at nodes indicate levels of bootstrap support (%) based on analysis of 1000 resampled datasets; only values above 50% are given. NCBI accession numbers for each sequence are provided in parentheses.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific purified proteins, or to particular nucleic acids, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a purified polypeptide” includes mixtures of two or more purified polypeptides.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not. For example, the phrase “the medium can optionally contain glucose” means that the medium may or may not contain glucose as an ingredient and that the description includes both media containing glucose and media not containing glucose.

Provided herein is an isolated Gram-positive non-motile, non-spore-forming, aerobic bacterium, wherein the bacterium produces a bacteriocin that kills or inhibits the growth of Gram-positive or Gram-negative target bacteria. An example of the bacterium is designated Streptomyces scopuloiridis, having the ARS Culture Collection No. NRRL B-24574^{T}, and the DSMZ general collection accession number DSM 41917. As used herein, an “isolated bacterium” is a bacterium of a single species that is not mixed with other bacterial species. As used herein, “bacteriocin” means a ribosomally synthesized protein or polypeptide produced by a bacterium that kills or inhibits the growth of target bacteria. As used herein, “target bacteria” are bacteria intended to be killed or whose growth is intended to be inhibited, for example by a bacteriocin.

The disclosed bacterium exhibits a range of chemotaxonomic and phenotypic characters typical of the members of the genus Streptomyces (Table 1). Strain RB72 forms an extensively branched substrate mycelium and aerial hyphae on several standard growth media. The disclosed bacterium produces white aerial hyphae with no spores and a golden brown substrate mycelium on all standard morphological media tested with the exception of International Streptomyces Project medium 2 (ISP2), on which the extent of the aerial hyphae formation is reduced and the substrate mycelium does not produce pigment. Sporulation of the aerial hyphae is not detected after prolonged growth periods, and the aerial hyphae remain white in color, typical of other Streptomyces strains that do not sporulate [28, 29, 30, 31, 32]. These results suggest a deficiency in the whi pathway of the organism.

In one aspect, the bacterium comprises a 16S rRNA nucleic acid that is greater than 98.87% identical to the nucleic acid sequence identified as SEQ ID NO:1, wherein the nucleic acid sequence is deposited under GenBank Accession No. EF657884. In yet another aspect, the bacterium comprises a 16S rRNA nucleic acid identified as SEQ ID NO:1.

In one aspect, the bacterium produces a bacteriocin comprising a polypeptide comprising a 15 amino acid sequence identified as SEQ ID NO:2, wherein the polypeptide has a weight of about 27 kDa as visualized by SDS-PAGE.

Further provided is an isolated polypeptide comprising a 15 amino acid sequence identified as SEQ ID NO:2, wherein the polypeptide has a weight of about 27 kDa as visualized by SDS-PAGE, wherein the polypeptide, and/or fragments thereof, kill or inhibit the growth of Gram-positive or Gram-negative target bacteria.

Examples of Gram-positive target bacteria include, but are not limited to, Streptomyces avermitilis, Streptomyces coelicolor, Streptomyces lividans, Streptomyces venezuelae, Nocardia salmonicida, Nocardia vaccinii, Rhodococcus marinonascens, Bacillus megaterium, Bacillus subtilis, Bacillus cereus, Enterococcus faecalis, Micrococcus luteus, Staphylococcus aureus, Streptococcus sp., Streptococcus pyogenes, Listeria monocytogenes, Clostridium perfringens, Clostridium botulinum, Lactococcus cremoris, Lactobacillus sp., and Leuconostoc sp. Thus, a Gram-positive target bacterium can be selected from the group consisting of Streptomyces avermitilis, Streptomyces coelicolor, Streptomyces lividans, Streptomyces venezuelae, Nocardia salmonicida, Nocardia vaccinii, Rhodococcus marinonascens, Bacillus megaterium, Bacillus subtilis, Bacillus cereus, Enterococcus faecalis, Micrococcus luteus, Staphylococcus aureus, Streptococcus sp., Streptococcus pyogenes, Listeria monocytogenes, Clostridium perfringens, Clostridium botulinum, Lactococcus cremoris, Lactobacillus sp., and Leuconostoc sp

Examples of Gram-negative target bacteria include, but are not limited to, Escherichia coli DH10B, Klebsiella pneumoniae, Salmonella typhimurium, Salmonella enteritidis, Campylobacter jejuni, Yersinia enterocolitica, Yersinia pseudotuberculosis, Vibrio cholerae O1 and non-O1, Vibrio parahaemolyticus, Vibrio vulnificus, Aeromonas hydrophila, Plesiomonas shigelloides, Shigella sonnei, Shigella flexneri, Enterobacter aerogenes, Flavobacterium sp., Acinetobacter sp., and Proteus sp. Thus, a Gram-negative target bacterium can be selected from the group consisting of Escherichia coli DH10B, Klebsiella pneumoniae, Salmonella typhimurium, Salmonella enteritidis, Campylobacter jejuni, Yersinia enterocolitica, Yersinia pseudotuberculosis, Vibrio cholerae O1 and non-O1, Vibrio parahaemolyticus, Vibrio vulnificus, Aeromonas hydrophila, Plesiomonas shigelloides, Shigella sonnei, Shigella flexneri, Enterobacter aerogenes, Flavobacterium sp., Acinetobacter sp., and Proteus sp.

In one aspect, the polypeptide and fragments thereof retain activity when stored at a temperature from about −20° C. to about 60° C. Thus, the polypeptide and fragments thereof can retain activity when stored at a temperature about −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C., or any intervening temperatures. As used herein, “retains activity” means retaining the ability to kill or inhibit the growth of Gram-positive or Gram-negative target bacteria.

In another aspect, the polypeptide and fragments thereof kill or inhibit the growth of target bacteria at a temperature about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C., or any intervening temperatures.

In another aspect, the polypeptide and fragments thereof retain activity when stored at a pH from about 3 to about 9. Thus, the polypeptide and fragments thereof retain activity when stored at a pH of about 3, 4, 5, 6, 7, 8, or 9, or any intervening pH value.

In a further aspect, the polypeptide and fragments thereof kill or inhibit the growth of Gram-positive or Gram-negative target bacteria at a pH of about 7.

Further provided is an isolated nucleic acid comprising a nucleotide sequence identified as SEQ ID NO:3, wherein the nucleic acid encodes a polypeptide comprising a 15 amino acid sequence identified as SEQ ID NO:2, wherein the polypeptide has a weight of about 27 kDa as visualized by SDS-PAGE, wherein the polypeptide kills or inhibits the growth of Gram-positive or Gram-negative target bacteria. Also provided are fragments of the nucleic acid identified as SEQ ID NO:3 which encode fragments of the polypeptide having an amino acid sequence identified as SEQ ID NO:2.

Further provided is a composition comprising the disclosed polypeptide and/or fragments and a carrier. As used herein, a “carrier” is any substance that can be used to facilitate and/or potentiate the activity of the disclosed polypeptide and/or fragments thereof for killing or inhibiting the growth of Gram-positive and Gram-negative target bacteria. Examples of carriers include but are not limited to calcium alginate, cellulose, cellophane, collagen casings, corn zein, gelatin, silicon coatings, nylon, polyethylene, soy protein, and other polymer plastic films.

Further provided is a method of killing or inhibiting the growth of a Gram-positive or Gram-negative target bacterium, comprising contacting the target bacterium with an effective amount of a bacteriocin comprising the disclosed polypeptide, and/or fragments thereof, whereby contacting the target bacterium with the bacteriocin kills or inhibits the growth of the target bacterium. As used herein, an “effective amount” is that amount which can bring about a desired result without causing adverse effects and is within the knowledge of one skilled in the art. Various methods are known by which a person of skill can determine the effective amount of bacteriocin required to kill or inhibit the growth of a Gram-positive or Gram-negative target bacterium.

Target bacteria can be selected from the group consisting of Streptomyces avermitilis, Streptomyces coelicolor, Streptomyces lividans, Streptomyces venezuelae, Nocardia salmonicida, Nocardia vaccinii, Rhodococcus marinonascens, Bacillus megaterium, Bacillus subtilis, Bacillus cereus, Enterococcus faecalis, Micrococcus luteus, Staphylococcus aureus, Streptococcus sp., Streptococcus pyogenes, Listeria monocytogenes, Clostridium perfringens, Clostridium botulinum, Lactococcus cremoris, Lactobacillus sp., Leuconostoc sp., Escherichia coli DH10B, Klebsiella pneumoniae, Salmonella typhimurium, Salmonella enteritidis, Campylobacter jejuni, Yersinia enterocolitica, Yersinia pseudotuberculosis, Vibrio cholerae O1 and non-O1, Vibrio parahaemolyticus, Vibrio vulnificus, Aeromonas hydrophila, Plesiomonas shigelloides, Shigella sonnei, Shigella flexneri, Enterobacter aerogenes, Flavobacterium sp., Acinetobacter sp., and Proteus sp.

Also provided is a method of growing a Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria, comprising culturing the bacterium in a medium and at a temperature effective to promote growth of the bacterium. Examples of media on which the disclosed bacterium can grow include, but are not limited to, mannitol soya flour agar, nutrient agar with 0.4% dextrose, yeast extract-malt extract agar (International Streptomyces Project medium 2, (ISP2)), and oatmeal agar (International Streptomyces Project medium 3, (ISP3)). Further, the disclosed bacterium can grow in liquid cultures of the same respective recipes that do not have an agar gelling agent. An exemplary temperature at which the disclosed bacterium can grow is about 25° C.

In another aspect, provided is a method of producing the disclosed polypeptide, and/or fragments thereof, comprising: (a) culturing a bacterium producing the polypeptide, and/or fragments thereof, under conditions suitable to produce the polypeptide, and/or fragments thereof; and (b) isolating the polypeptide, and/or fragments thereof, from the bacterium.

In another aspect, provided is a method of producing the disclosed polypeptide, and/or fragments thereof, comprising: (a) synthesizing the polypeptide, and/or fragments thereof, in a reaction mixture under conditions suitable to produce the polypeptide, and/or fragments thereof; and (b) isolating the polypeptide, and/or fragments thereof, from the reaction mixture.

In another aspect, provided is a method of producing the disclosed polypeptide, and/or fragments thereof, comprising: (a) expressing the polypeptide and/or fragments thereof, in a recombinant expression system; and (b) isolating the polypeptide and/or fragments thereof, from the expression system.

In another aspect, provided is a method of preserving food or preventing bacterial contamination of food, comprising contacting the food with an effective amount of a bacteriocin comprising a polypeptide, and/or a fragment thereof, wherein the polypeptide comprises a 15 amino acid sequence identified as SEQ ID NO:2, wherein the polypeptide has a weight of about 27 kDa as visualized by SDS-PAGE, wherein the polypeptide, and/or fragments thereof, kill or inhibit the growth of Gram-positive or Gram-negative target bacteria, whereby contacting the food with the bacteriocin preserves the food or prevents bacterial contamination of the food. A person of skill would know how to contact food in order to preserve the food or prevent bacterial contamination of the food. For example, the disclosed bacteriocin can be isolated from the disclosed Streptomyces species and can then be mixed with a food, for example, ground meat, sauces, or milk. Further, the disclosed bacteriocin can be applied to the surface of a food, for example, fruits, vegetables, meat, or poultry. In another aspect, foods can be inoculated with the disclosed bacterium that produces the bacteriocin in the food. In yet another aspect, a mixture of the disclosed bacteriocin and the disclosed bacterium that produces the bacteriocin can be added to food. In another aspect, the disclosed bacteriocin can be added to the food bound to a carrier, so that the carrier can act as a reservoir of the bacteriocin. In yet another aspect, the disclosed bacteriocin can be immobilized onto a material that is applied to the surface of the food. Examples of materials that the disclosed bacteriocin can be incorporated into or immobilized upon include, but are not limited to, calcium alginate, cellulose, cellophane, collagen casings, corn zein, gelatin, silicon coatings, nylon, polyethylene, soy protein, and other polymer plastic films. For example, the inner surface of polyethylene packaging for frankfurters can contain the disclosed bacteriocin to kill or inhibit the growth of Gram-positive or Gram-negative target bacteria during storage of the frankfurters.

In another aspect, provided is a method of preventing bacterial contamination of a surface of an object, comprising contacting the surface of the object with an effective amount of a bacteriocin comprising the disclosed polypeptide, and/or fragments thereof, whereby contacting the surface of the object with the bacteriocin prevents bacterial contamination of the surface of the object. Examples of surfaces include, but are not limited to, food, water, toys, operating tables, catheters, wound dressings, bandages, and pens.

In another aspect, provided is a method of killing or inhibiting the growth of Gram-positive or Gram-negative target bacteria on a surface of an object, comprising contacting the target bacteria with an effective amount of a bacteriocin comprising the disclosed polypeptide, and fragments thereof, whereby contacting the target bacteria on the surface of the object kills or inhibits the growth of the target bacteria. Examples of surfaces include, but are not limited to food, water, toys, operating tables, catheters, wound dressings, bandages, and pens.

As used herein, a “purified polypeptide” or “isolated polypeptide” is a polypeptide that is substantially free from the materials with which the polypeptide is normally associated in nature or in culture. The polypeptides, or fragments thereof, can be obtained, for example, by extraction from a natural source, for example, an isolated Gram-positive non-motile, non-spore-forming, aerobic bacterium that produces a bacteriocin that kills or inhibits the growth of Gram-positive or Gram-negative target bacteria; by expression of a recombinant nucleic acid encoding the polypeptide (for example, in a cell or in a cell-free translation system); or by chemically synthesizing the polypeptide. In addition, a polypeptide may be obtained by cleaving full-length polypeptides. When the polypeptide is a fragment of a larger naturally occurring polypeptide, the purified (isolated) polypeptide is shorter than and excludes the full-length, naturally-occurring polypeptide of which it is a fragment.

The disclosed polypeptides can be prepared by using any of a number of chemical polypeptide synthesis techniques well-known to those of ordinary skill in the art, including solution methods and solid phase methods. One method of producing the disclosed polypeptides is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry (Applied Biosystems, Inc., Foster City, Calif.). One skilled in the art can readily appreciate that a peptide or polypeptide corresponding to a bacteriocin of the present invention, for example, can be synthesized by standard chemical reactions.

For example, enzymatic ligation of cloned or synthetic peptide segments allows relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intra-molecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The disclosed polypeptides, and fragments thereof, can also be prepared by other means including, for example, recombinant techniques. Examples of appropriate cloning and sequencing techniques, and instructions sufficient to direct persons of skill through many cloning exercises are found in Sambrook et al. (2001) Molecular Cloning—A Laboratory Manual (3rd ed.) Vol. 1-3, Cold Spring Harbor Laboratory, Cold Spring Harbor Press, NY, (Sambrook).

It is understood that as discussed herein, the terms “similar” or “similarity” mean the same thing as “homology” and “identity.” Thus, for example, if the use of the word homology is used to refer to two non-natural sequences, it is understood that this is not necessarily indicating an evolutionary relationship between these two sequences, but rather is looking at the similarity or relatedness between their nucleic acid or amino acid sequences. Many of the methods for determining similarity between two evolutionarily related molecules are routinely applied to any two or more nucleic acids or polypeptides for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of similarity, or homology, to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity, or homology, to the stated sequence or the native sequence. Those of skill in the art readily understand how to determine the similarity of two polypeptides or nucleic acids. For example, the similarity can be calculated after aligning the two sequences so that the similarity is at its highest level.

Another way of calculating similarity, or homology, can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA) in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html)), or by inspection.

The same types of similarity, or homology, can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if similarity is found with at least one of these methods, the sequences would be said to have the stated similarity.

For example, as used herein, a sequence recited as having a particular percent similarity, homology, or identity to another sequence refers to sequences that have the recited similarity as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of the calculation methods (although, in practice, the different calculation methods will often result in different calculated similarity percentages).

Each of the disclosed polypeptides, and fragments thereof, can have one or more conservative amino acid substitutions. These conservative substitutions are such that a naturally occurring amino acid is replaced by one having similar properties. Such conservative substitutions do not alter the function of the polypeptide. For example, conservative substitutions can be made according to Table 4, shown below.

Thus, it is understood that, where desired, modifications and changes may be made in the nucleic acid encoding the polypeptides of this invention and/or in the amino acid sequence of the disclosed polypeptides and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity.

Further provided is an isolated nucleic acid that encodes the disclosed purified polypeptides and variants or fragments thereof. An example of a disclosed nucleic acid that encodes the disclosed polypeptide includes the nucleic acid identified as SEQ ID NO:3.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in Sambrook et al. (2001) Molecular Cloning—A Laboratory Manual (3rd ed.) Vol. 1-3, Cold Spring Harbor Laboratory, Cold Spring Harbor Press, NY, (Sambrook).

The nucleic acids of this invention can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. Thus, the probe of this invention can be either DNA or RNA and can bind either DNA or RNA, or both, in the biological sample. The nucleic acids of the present invention, for example SEQ ID NO:3, and fragments thereof, can be utilized as probes or primers to detect nucleic acids of the disclosed bacterium. A polynucleotide probe or primer comprising at least 15 contiguous nucleotides can be utilized to detect a nucleic acid of the disclosed bacterium. Therefore, the polynucleotide probes or primers of this invention can be at least 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or at least 200 nucleotides in length.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

“Stringent conditions” refers to the washing conditions used in a hybridization protocol. In general, the washing conditions should be a combination of temperature and salt concentration chosen so that the denaturation temperature is approximately 5° C. to 20° C. below the calculated Tₘ of the nucleic acid hybrid under study. The temperature and salt conditions are readily determined empirically in preliminary experiments in which samples of reference DNA immobilized on filters are hybridized to the probe or polypeptide-coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C. Stringent conditions are known to one of skill in the art. See, for example, Sambrook et al. (2001). The following is an exemplary set of hybridization conditions and is not limiting:

### Very High Stringency

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

### High Stringency

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

### Low Stringency

| | |
|---|---|
| Hybridization: | 6x SSC at RT to 55° C. for 16-20 hours |
| Wash at least twice: | 2x-3x SSC at RT to 55° C. for 20-30 minutes each. |

As mentioned above, the disclosed nucleic acids and fragments thereof can be utilized as primers to amplify a disclosed bacterial nucleic acid by standard amplification techniques. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195; 4,683,202; and 4,965,188. Each of these publications is incorporated herein by reference in its entirety for PCR methods. One of skill in the art would know how to design and synthesize primers that amplify the disclosed nucleic acids or fragments thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H, etc. The label may be a two stage system, where the amplified DNA is conjugated to biotin, haptens, etc., having a high affinity binding partner, e.g., avidin, specific antibodies, etc., where the binding partner is conjugated to a detectable label. The label may be conjugated to one or both of the primers. Alternatively, the pool of nucleotides used in the amplification is labeled so as to incorporate the label into the amplification product.

The sample nucleic acid, for example amplified fragment, can be analyzed by one of a number of methods known in the art. The nucleic acid can be sequenced by dideoxy or other methods. Hybridization with the sequence can also be used to determine its presence, by Southern blots, dot blots, etc.

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

In another aspect, provided is a vector, comprising a disclosed nucleic acid. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.). The vector, for example, can be a plasmid. The vectors can contain genes conferring hygromycin resistance, gentamicin resistance, or other genes or phenotypes suitable for use as selectable markers, or methotrexate resistance for gene amplification.

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences, for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures. Also, nucleic acid modifications can be made to promote amino terminal homogeneity.

Additionally, yeast expression can be used. The invention provides a nucleic acid encoding a polypeptide of the present invention, wherein the nucleic acid can be expressed by a yeast cell. More specifically, the nucleic acid can be expressed by Pichia pastoris or S. cerevisiae. There are several advantages to yeast expression systems, which include, for example, Saccharomyces cerevisiae and Pichia pastoris. First, evidence exists that proteins produced in a yeast secretion systems exhibit correct disulfide pairing. Second, efficient large scale production can be carried out using yeast expression systems. The Saccharomyces cerevisiae pre-pro-alpha mating factor leader region (encoded by the MFα-1 gene) can be used to direct protein secretion from yeast (Brake, et al.). The leader region of pre-pro-alpha mating factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha mating factor leader region. This construct can be put under the control of a strong transcription promoter, such as the alcohol dehydrogenase I promoter, alcohol oxidase I promoter, a glycolytic promoter, or a promoter for the galactose utilization pathway. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

In another aspect, provided are vectors containing the disclosed nucleic acids in a host suitable for expressing the nucleic acids. The host cell can be a prokaryotic cell, including, for example, a bacterial cell. In one aspect, the bacterial cell can be an isolated Gram-positive bacterium, wherein the bacterium is a non-motile, non-spore-forming, aerobic bacterium, wherein the bacterium produces a bacteriocin that kills or inhibits the growth of Gram-positive or Gram-negative target bacteria. Moreover, the bacterial cell can be an E. coli cell.

Alternatively, the cell can be a eukaryotic cell, including, for example, a Chinese hamster ovary (CHO) cell, a myeloma cell, a Pichia cell, or an insect cell. The coding sequence for any of the polypeptides described herein can be introduced into a Chinese hamster ovary (CHO) cell line, for example, using a methotrexate resistance-encoding vector, or other cell lines using suitable selection markers. Presence of the vector DNA in transformed cells can be confirmed by Southern blot analysis or PCR. Production of RNA corresponding to the insert coding sequence can be confirmed by Northern blot analysis or RT-PCR. A number of other suitable host cell lines have been developed and include myeloma cell lines, fibroblast cell lines, and a variety of tumor cell lines such as melanoma cell lines. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

Further provided is a method of making any of the disclosed purified polypeptides, fragments and variants described herein comprising culturing a host cell comprising a vector that encodes a polypeptide and purifying the polypeptide produced by the host cell. As mentioned above, these polypeptides include, but are not limited to, the bacteriocin polypeptide, fragments thereof, polypeptides comprising an amino acid sequence similar to the disclosed amino acid sequences or fragments thereof and the purified bacteriocin polypeptide, fragments thereof, with one or more conservative amino acid substitutions.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Bacterial Strains and media. The bacterial strains used in this study are described in Table 2. Stock cultures of all strains were stored in nutrient broth (NB) with 0.4% dextrose and 20% glycerol at −80° C. Strains were subcultured onto nutrient agar (NA) with 0.4% dextrose for working cultures. Streptomyces strains in regular use were stored as spore suspensions in 20% glycerol at −20° C. and maintained as actively growing cultures on mannitol soya flour medium [33]. Strains of Streptomyces were incubated at 30° C. All other strains used were grown under their respective optimal growth conditions.

The BLIS RB72 producer strain was isolated from soil collected at Rainbow Bluff, a woodland rock outcropping in Lynn, Ala. The bacterium was cultivated from a soil sample suspension inoculated on an agar medium designed from a 1:1 dilution of a cold-water extraction (1 l soil/1 l water; incubated for 3 days at 4° C.) of the organism's native soil. The medium was supplemented with cycloheximide (10 μg/ml), nalidixic acid (20 μg/ml), and catalase (100 U/ml). After incubation for 14 days at 25° C., the colony was selected for further isolation on nutrient agar. It was classified as a member of the genus Streptomyces by analysis of its partial 16S rRNA gene sequence (GenBank accession number EF657884), phenotypic, and chemotaxonomic characters.

Preparation of the Streptomyces sp. strain RB72 bacteriocin BLIS RB72. Batch cultures (2-5 liters) of Streptomyces sp. strain RB72 were grown for 72 h in NB with 0.4% dextrose at 30° C. with shaking. Cells were removed from the culture supernatant by centrifugation at 10,000×g for 30 mM followed by passage through a 0.22 μm CN vacuum filter system (Corning, Inc., Corning, N.Y.). The bacteriocin was concentrated in the retentate (500 ml) with a 10,000 molecular-weight cutoff membrane in a stirred ultrafiltration cell (Diaflo; Amicon, Inc., Beverly, Mass.).

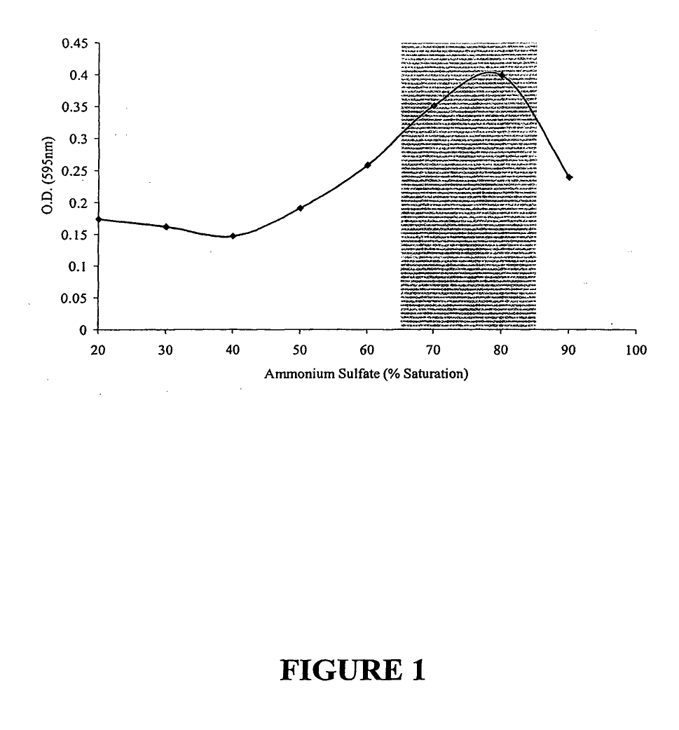

BLIS RB72 was best recovered from the supernatant at 80% saturation during an ammonium sulfate gradient precipitation of the strain RB72 culture supernatant (FIG. 1). The ammonium sulfate purification fraction (ASPF) of the retentate was initiated by a 65%-saturated precipitation of undesired proteins. Precipitation proceeded for 1 h on ice with gentle stirring, and the precipitated protein was removed from the retentate by centrifugation at 10,000×g for 20 min at 4° C. The bacteriocin was recovered from the retentate by addition of ammonium sulfate to a final saturation of 90%, precipitated at 4° C. overnight, and collected by centrifugation at 10,000×g for 20 min at 4° C.

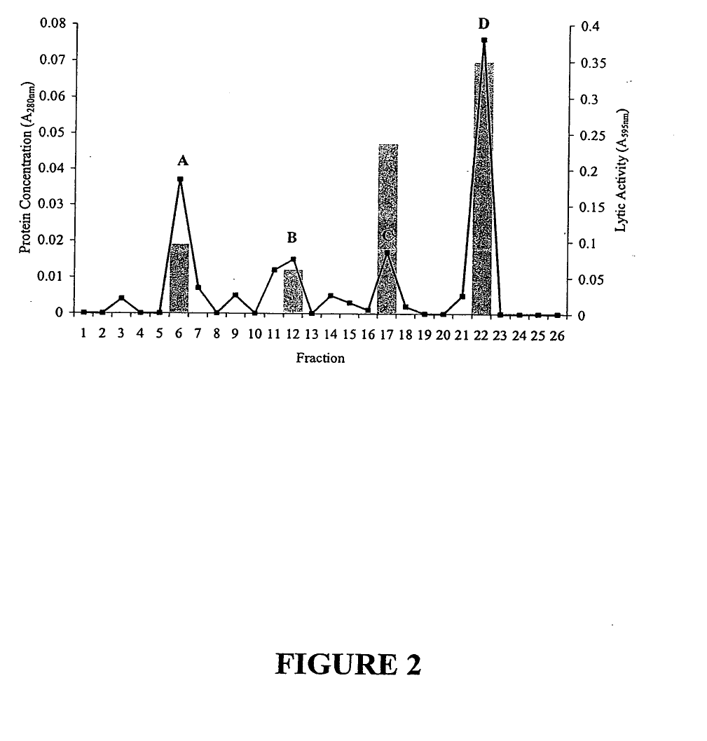

The bacteriocin was dissolved in 100 ml DEAE ion exchange start buffer (IEB; 100 mM Tris, 10 mM EDTA, pH 8.2). Residual ammonium sulfate was removed by dialysis in three changes of IEB at 4° C. with gentle mixing over 24 h. This preparation was labeled as ASPF-purified bacteriocin. Fifty milliliters of this preparation were applied to a DEAF Sepharose® anion exchange column (Sigma-Aldrich, St. Louis, Mo.) equilibrated with IEB at 4° C. Unbound proteins were washed from the column with IEB until absorbance of the column exudates at 280 nm (A₂₈₀) using a SmartSpec 3000 spectrophotometer (Bio-Rad Laboratories, Inc., Hercules, Calif.) returned to 0. IEB containing 250 mM NaCl was passed through the column at a flow rate of 1 ml/min and fractions were collected until the A₂₈₀ returned to baseline. Remaining bound proteins, which included the major bacteriocin activity as shown by a NaCl gradient assay (FIG. 2), were removed from the column by passing IEB containing 2M NaCl through the column at a flow rate of 1 ml/min. Fractions were collected until the A₂₈₀ returned to baseline. The 2M NaCl fraction was concentrated to 10 ml, desalted, and buffer exchanged with dye-release assay buffer using a Centriplus YM10 centrifugal filter unit (Millipore, Billerica, Mass.) at 4° C. with 3000×g centrifugation.

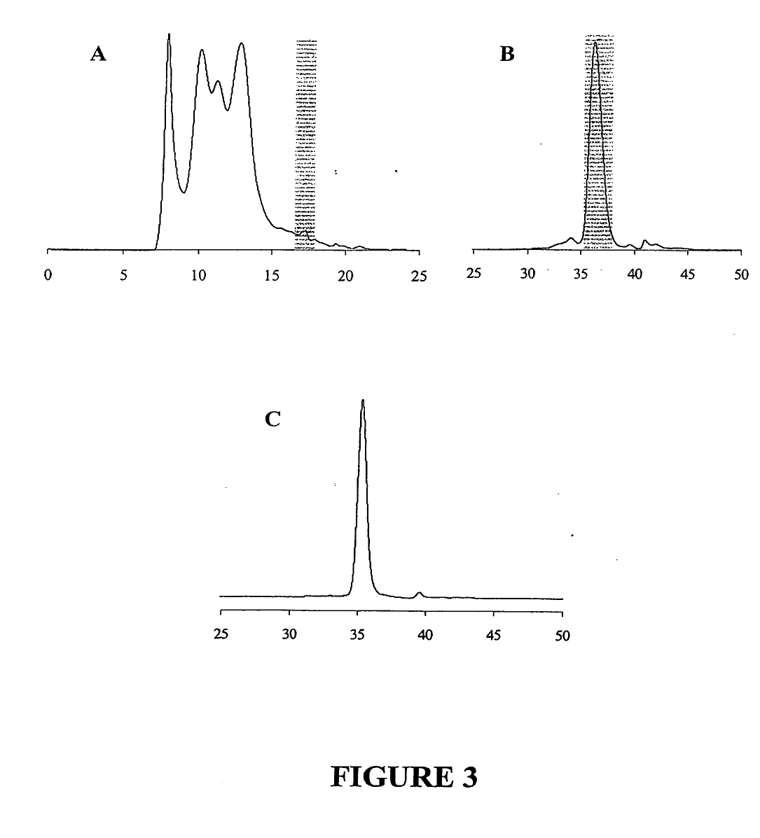

For purification by fast protein liquid chromatography (FPLC), aliquots of 300 μA were loaded onto a Superose™ 12 gel filtration column (GE Healthcare Bio-Sciences, Corp., Piscataway, N.J.), equilibrated and run at 1 ml/min, in 25 mM ammonium acetate buffer (pH 6.5) using a Shimadzu LC-10ATvp liquid chromatograph (Shimadzu Scientific Instruments, Columbia, Md.). The runs were monitored for A₂₈₀ using a Shimadzu SPD-M10Avp diode array detector, and the active fractions of multiple runs were collected using a Shimadzu FRC-10A fraction collector. The fractions were pooled, lyophilized, and resuspended in ammonium acetate buffer. A second-round of gel filtration at a flow rate of 0.5 ml/min allowed collection of the bacteriocin at 35 min (FIG. 3). The active fraction was pooled, lyophilized, and stored at −20° C. At each point of the purification process and for determining active fractions, bacteriocin activity was assayed using a dye-release assay.

Dye-release enzyme detection assay. The presence of the bacteriolytic enzyme was rapidly assessed using a modified version of the dye-release assay described by Zhou et al. [34]. S. coelicolor, grown to mid-exponential phase in one liter of NB with 0.4% dextrose, was heat-killed and washed 3× with EP water (distilled water purified with an E-Pure system: Barnstead International, Dubuque, Iowa). The cell substrate (0.5 g wet wt/30 ml solution) was stained in a reaction solution of 20 mM Remazol brilliant blue R (Sigma-Aldrich, St. Louis, Mo.) and 250 mM NaOH. The substrate-staining reaction solution was incubated with gentle mixing for 6 h at 37° C. and then for 12 h at 4° C. Dyed cell substrates were harvested by centrifugation at 5000×g for 20 min, washed with several changes of EP water until no unbound dye was released from the substrate, and stored at −20° C.

The substrate was washed 2× with assay buffer (25 mM Tris-HCl, 10 mM NaCl, pH 7.2) prior to use in the dye-release assay. The dye-labeled cell suspension was standardized to an optical density of 2.0 at 595 nm. At each step of the bacteriocin purification process, 50 μl aliquots of the protein separation were added to 200 μl of the standardized dye-labeled cell suspension. The mixtures were incubated at 37° C. for 24 h with continual oscillation. The reactions were arrested by the addition of 25 μl of ethanol, and undigested substrate was removed from the supernatant by centrifugation at 5000×g for 5 min. The supernatants of each reaction mixture were transferred to a flat-bottom microplate for qualitative comparison to the negative control or for quantitative comparison of absorbance measurements at 595 nm using a μQuant microplate spectrophotometer (BioTek Instruments, Inc., Winooski, Vt.).

Spectrum of BLIS RB72 bacteriolytic activity. The bacteriolytic activity of Streptomyces sp. strain RB72 against all indicator strains was detected as a zone of lysis around the growth of strain RB72 on NA with 0.4% dextrose that contained a heat-killed indicator organism (Table 2). The antimicrobial activity of the ammonium sulfate precipitation fraction (ASPF)-purified bacteriocin preparation was assessed using a quantitative dye-release assay and was expressed as arbitrary units (AU). By definition, 1 AU results in a 0.1 increase in the optical density of the dye-release reaction supernatant at 595 nm after incubation.

Effects of proteolytic enzymes, temperature, and pH on bacteriocin activity. The effects of proteolytic enzymes on the activity of the bacteriocin were tested using 150 AU of ASPF-purified bacteriocin in dye-release assay buffer. The proteolytic enzymes used, as described by Risoen et al. [35], were α-chymotrypsin (1 mg/ml), trypsin (1 mg/ml), and proteinase K (10 mg/ml). Residual bacteriocin activity was measured using a quantitative dye-release assay after incubation in the presence of each proteolytic enzyme for 4 h at 37° C. Residual activity was totaled by deducting the results of the dye-release assay for the proteolytic enzyme incubated in buffer alone from the activity measured for the BLIS RB72 exposed to the proteolytic enzyme. A 150 AU ASPF-purified bacteriocin suspension not subjected to proteolytic activity but incubated for 4 h at 37° C. was measured as a mark of unaltered bacteriocin activity.

Thermal stability of the bacteriocin was analyzed by incubating 30 AU of ASPF-purified bacteriocin in dye-release assay buffer at temperatures and times described in Table 3. Residual bacteriocin activity was assessed using a quantitative dye-release assay.

Stability of ASPF-purified bacteriocin (150 AU) against various pH values (pH 2-9) for 4 h at 4° C. was assessed by neutralizing each suspension to pH 7.0 after incubation and measuring residual bacteriocin activity using a quantitative dye-release assay.

Estimation of protein concentration. The microplate assay protocol of the DC Protein Assay (Bio-Rad Laboratories, Inc., Hercules, Calif.) was used to estimate protein concentration. Standards were made by diluting bovine serum albumin in EP water to concentrations between 0.1 and 1.0 mg/ml. The optical density of each developed reaction was measured at 750 nm in a flat-bottom microtiter plate using a microplate spectrophotometer.

Analysis of purified bacteriocin. The purity of the bacteriocin was assessed using sodium dodecyl sulfate-polyacrylamide gel electrophoresis (SDS-PAGE [36]) using 15% and 20% resolving gels and a Mini-Protean II electrophoresis system (Bio-Rad Laboratories, Inc., Hercules, Calif.). Buffers and reagents were as described by Sambrook and Russell [37]. Proteins were visualized after staining with Bio-Safe coomassie stain (Bio-Rad Laboratories, Inc., Hercules, Calif.). SDS-PAGE zymograms were performed as described by Neumann et al. [38] using heat-killed S. coelicolor as substrate to detect bacteriocin activity in 15% polyacrylamide gels. SDS was removed from the zymograms with repeated washes in distilled water and bacteriocin activity was detected by incubating the gels in lytic assay buffer [38] at 30° C. for 36 h with gentle mixing. After incubation. the gels were stained with 0.1% methylene blue chloride in 10 mM potassium hydroxide for 20 min and destained in several changes of distilled water. Images of the SDS-PAGE and SDS-PAGE zymogram gels were photographed with a Fotodyne (FOTO/Analyst Investigator) imaging system (Fotodyne, Inc., Hartland, Wis.).

Effect of BLIS RB72 on the viability of Bacillus subtilis 168 indicator culture. The viability of the indicator organism incubated in the presence of the bacteriocin over time was assessed with modifications of the method of Kamoun et al. [39]. Due to the clumping nature of S. coelicolor when grown in broth, Bacillus subtilis 168 was used as the indicator organism. An overnight culture of B. subtilis 168 was inoculated into 25 ml of fresh NB with 0.4% dextrose and incubated at 37° C. with shaking to exponential growth phase. The cells were harvested by centrifugation at 5000×g for 5 min, washed with three changes of 10 mM phosphate buffer (pH 7.0), and suspended in the same buffer at a concentration of 10⁷ cells/ml using a hemocytometer. One ml of this suspension was centrifuged at 5000×g for 5 min, and the cell pellet was resuspended in 1 ml prewarmed phosphate buffer containing 1500 AU ASPF-purified bacteriocin and incubated at 37° C. The viability of the indicator culture was monitored over 200 min. At each time point (0, 1, 5, 10, 20, 40, 80, 120, 160, and 200 min), 100 μl samples were removed, serially diluted to 10⁻⁶ in phosphate buffer, and plated onto NA with 0.4% dextrose. The plates were incubated overnight at 30° C. and colony forming units per milliliter (CFU/ml) were calculated.

Tandem mass spectral analysis of the purified bacteriocin. The purified BLIS RB72 protein was cut from a 15% SDS-PAGE gel, digested by trypsin, and analyzed by liquid chromatography tandem mass spectrometry at the University of California at Davis Proteomics Facility. The mass spectral data were analyzed in Mascot (Matrix Science, Inc., Boston, Mass.) to generate putative peptide sequences of BLIS RB72. The putative peptides were searched within the Mass Spectrometry Protein Sequence Database (MSDB) for potential matches to known protein sequences.

### Results

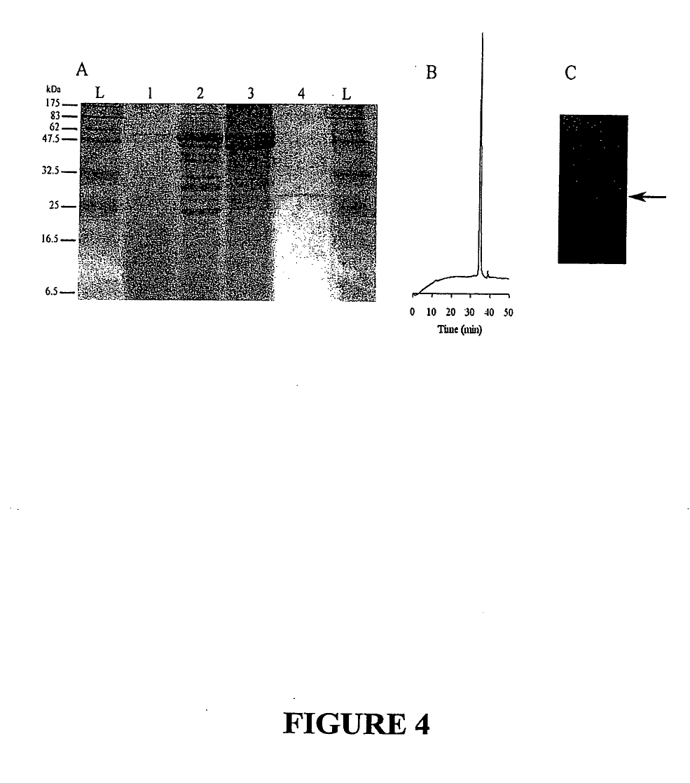

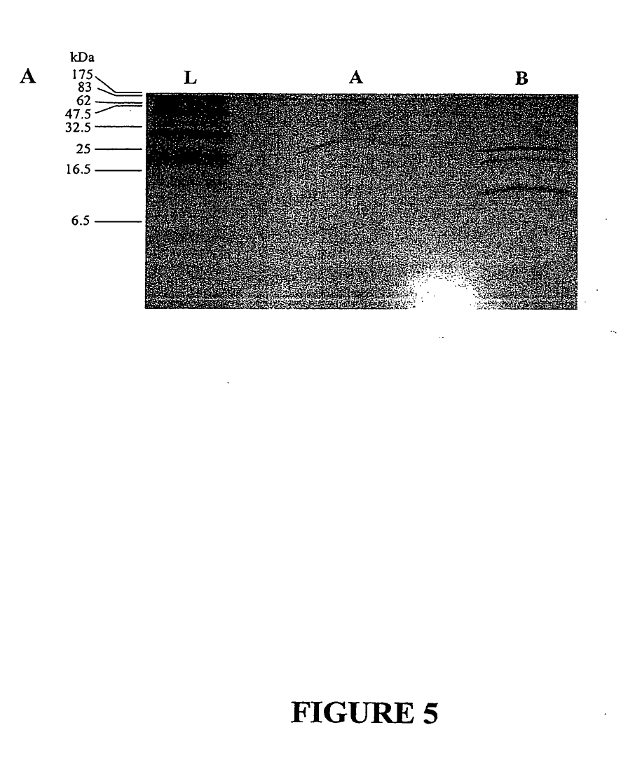

Preparation of the Streptomyces sp, strain RB72 bacteriocin BLIS RB72. The stepwise preparation of Streptomyces sp. strain RB72 bacteriocin yielded a purified enzyme as visualized by a SDS-PAGE gel containing 15% polyacrylamide (FIG. 4A) and as indicated by a single major chromatographic peak after two rounds of size exclusion FPLC (FIG. 4B). The purified enzyme also produced a single band (27 kDa) when separated using 20% polyacrylamide in a SDS-PAGE gel (FIG. 5, Lane A). The purified bacteriocin from the strain RB72 culture supernatant demonstrated lytic activity against S. coelicolor, which is visualized as a band of lysis in a SDS-PAGE zymograph (FIG. 4C).

Spectrum of BLIS RB72 bacteriolytic activity. The bacteriolytic activity of Streptomyces sp. strain RB72 was initially observed as zones of lysis surrounding colonies incubated in the presence of heat-killed S. coelicolor substrate incorporated into the bacteria growth medium. All Gram-positive and Gram-negative bacterial strains used as heat-killed substrate with the exception of the producer strain proved sensitive to the bacteriolytic enzyme produced by Streptomyces sp. strain RB72 (Table 2). Table 2 shows the spectrum of bacteriocin activity of the producer strain Streptomyces sp. strain RB72.

Effects of proteolytic enzymes, temperature, and pH on bacteriocin activity. ASPF-purified bacteriocin was sensitive to exposure to all proteolytic enzymes tested with the greatest sensitivity exhibited to proteinase K (Table 3). Table 3 shows the factors affecting the bacteriolytic activity of BLIS RB72. The enzyme exhibited thermostability below 40° C. and was increasingly inactivated above this temperature with complete inactivation at 80° C. (Table 3). The bacteriocin retained full activity following exposure to pH values of 7-9; however, the enzyme began to lose activity at pH values of 6-3 and was completely inactivated by incubation at pH 2 (Table 3).

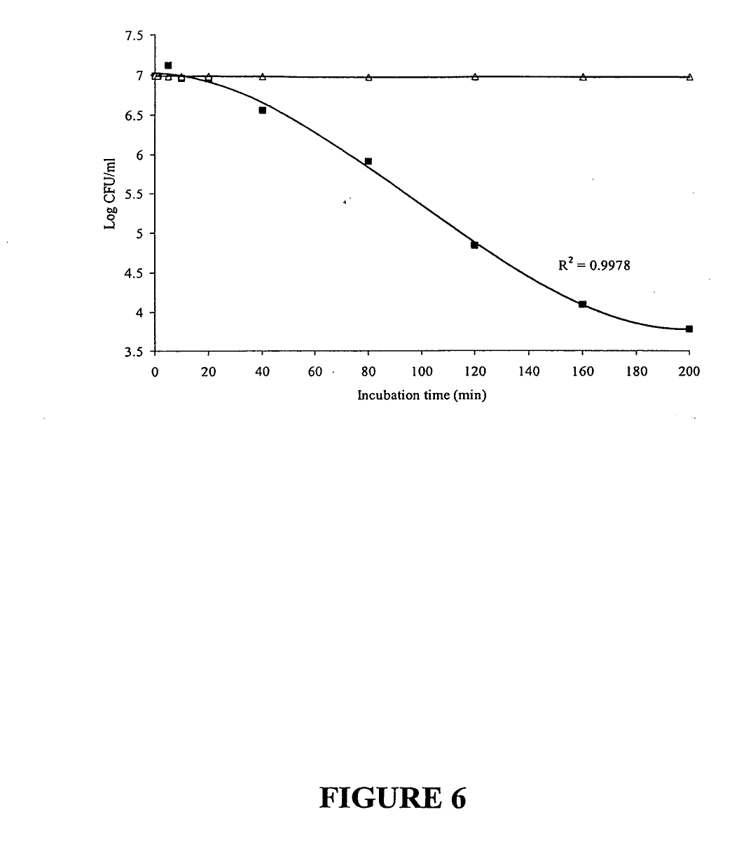

Effect of BLIS RB72 on the viability of Bacillus subtilis 168 indicator culture. The addition of ASPF-purified bacteriocin to B. subtilis 168 cells in the exponential growth phase resulted in a reduction of CFU/ml when compared to the CFU/ml of the cultures that received only buffer (FIG. 6). Following the addition of ASPF-purified bacteriocin, a decrease in viable count was observed after a 20 min incubation period.

Tandem mass spectral analysis of the purified bacteriocin. The spectral data generated from tandem mass spectrometry analysis of the trypsin-digested BLIS RB72 protein suggested a number of putative amino acid peptide fragments. One 15 amino acid peptide fragment, TALEDKAEGASIFQR (SEQ ID NO:2), showed homology to the peptide sequence of linocin M18, a predicted bacteriocin (GenBank accession number ZP_{—}01189115) from the genomic sequence of Halothermotrix orenii H168.

### Example 2

The morphological characteristics of strain RB72 were examined using light and scanning electron microscopy of colonies grown on mannitol soya flour agar, nutrient agar with 0.4% dextrose, yeast extract-malt extract agar (ISP medium 2), and oatmeal agar (ISP medium 3) after 7, 14, and 21 days at 25° C. The coverslip method of Hopwood [40] was used to observe the hyphal characters by phase-contrast light microscopy with a Nikon Eclipse E600 microscope (Nikon Instruments, Inc.) equipped with a Spot RT Color imaging system (version 3.4 imaging software; Diagnostic Instruments, Inc.). For high-resolution scanning electron microscopy, agar blocks containing mycelium were fixed with osmium tetroxide (1% wt/vol in 0.1M cacodylate buffer, pH 7.2) for 2 h, passed through increased concentrations of acetone (25, 50, 75, 90, 100%), and dried to critical point with a Denton DCP-1 critical point drying apparatus. The dried samples were mounted on graphite coated aluminum stubs, coated with gold/palladium alloy by a Technics hummer sputter coater, and examined with a Hitachi scanning electron microscope.

Colony morphology of strain RB72 was observed on several standard media (ISP2, ISP3, ISP4, ISP5) after 14 days incubation at 25° C. Examination of strain RB72 for a range of biochemical and physiological characters was as described by Shirting and Gottlieb [41], Williams et al. [42], and Kampfer et al. [43].

Genomic DNA was extracted from biomass of actively growing cultures on nutrient agar supplemented with 0.4% dextrose as described by Olson et al. [44]. PCR amplification using universal primers 24f and 1492r was performed as described by Farris and Olson [45]. Amplified fragments were ligated into pCR2.1 cloning vector (TA cloning kit; Invitrogen) and used to transform Escherichia coli DH10B (Invitrogen Corporation) according to the manufacturer's instructions. Plasmids with inserts of the correct size were sequenced at the Macrogen (Korea) sequencing facility.

Sequence data were aligned using Sequencher version 4.5 (Gene Codes Corporation), and relatedness to gene sequences of characterized Streptomyces was determined via NCBI/BLAST/blastn searches. The reference sequences and strain RB72 sequence (GenBank accession number EF657884) were aligned in BioEdit Sequence Alignment Editor, version 7.0.5.3 [46] using CLUSTAL W [47]. The neighbor-joining [48] algorithm of PAUP* version 4.0b 10 [49] was used to infer the phylogenetic relatedness of the sequences. The method of Kimura [50] was used to generate evolutionary distance matrices. Tree topologies were calculated by bootstrap analyses [51] based on 1000 re-samplings.

Results

The organism exhibited a range of chemotaxonomic and phenotypic characters typical of the members of the genus Streptomyces (Table 1). Table 1 shows a comparison of morphological, cultural, and physiological characteristics of strain RB72 and related Streptomyces species. The strains in Table 1 are: 1, strain RB72; 2, S. hachijoensis NBRC 12782^{T}; 3, S. mauvecolor NBRC 13854^{T}; 4, S. cyaneogriseus ATCC 27426^{T}; 5, S. xanthochromogenes NRRL B-54107^{T}; 6, S. microflavus NRRL B-2156^{T}. Data for type strain species are taken from Hatano et al. [52], Williams et al. [53], Kampfer et al. [54], and Bergy's Manual of Determinative Bacteriology [55]. SP, Spirales; RF, Rectiflexibiles; d, variable; +, positive; −, negative.

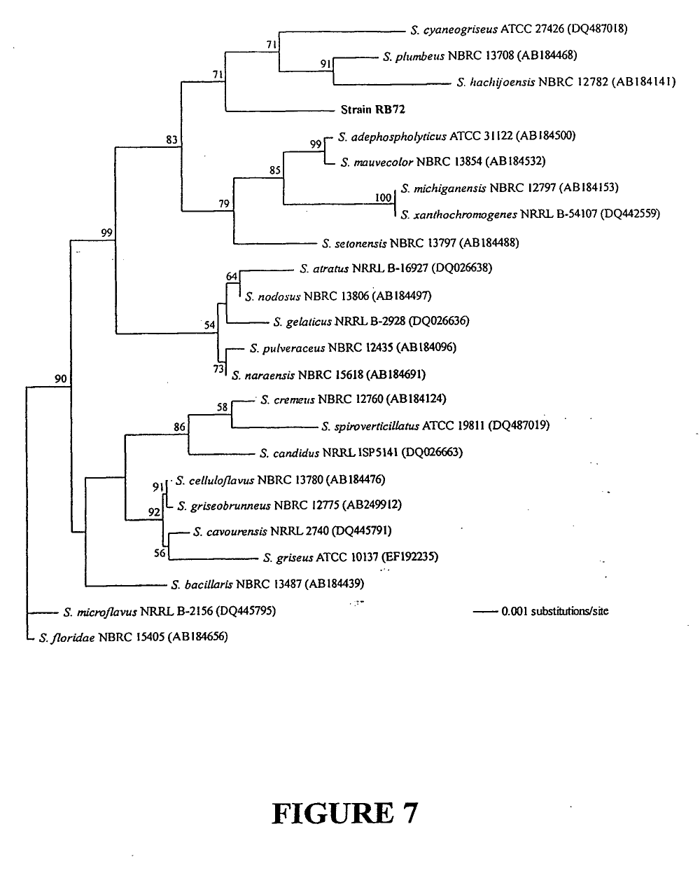

A near-complete 16S rRNA gene sequence (1332 nt) was determined for strain RB72. Comparison of the sequence with sequences of reference microorganisms confirmed that the unknown isolate was closely related to species of the genus Streptomyces. Phylogenetic analysis showed that strain RB72 formed a sister grouping with Streptomyces cyaneogriseus ATCC 27426^{T}, Streptomyces plumbeus NBRC 13708^{T}, and Streptomyces hachijoensis NBRC 12782^{T} (FIG. 7). Streptomyces hachijoensis NBRC 12782^{T} is currently in a state of re-evaluation and is considered a subjective synonym with Streptomyces cinnamoneus NBRC 12782^{T [}52]. Strain RB72 does not display the whorl-forming character observed in Streptomyces hachijoensis NBRC 12782^{T [}52], separating them phenotypically. Streptomyces cyaneogriseus ATCC 27426^{T} and Streptomyces plumbeus NBRC 13708^{T} were found to have the highest 16S rRNA gene sequence similarity values to strain RB72 at 98.87%.

Strain RB72 warrants the classification as the type strain of a novel species of the genus Streptomyces based on comparison of 16S rRNA gene sequence with other known Streptomyces species and the phenotypic characters of sole carbon source utilization, chemotaxonomic characters, broad spectrum bacteriocin production, and lack of sporulation that set it apart from other described Streptomyces. For strain RB72, the name Streptomyces scopuloiridis is proposed; sp. nov. (scop'u.lo.i.rid'is. L. masc. n. scopulus, cliff, bluff, crag; L. adj. suff. -o, originating from; L. fem. adj. Iridis, of or belonging to the goddess of the rainbow, referring to the location of isolation, Rainbow Bluff, a woodland bluff in Lynn, Ala.).

### REFERENCES

1. Tagg, J. R., A. S. Dajani, and I. W. Wannamaker. 1976. Bacteriocins of gram-positive bacteria. Microbiological Reviews 40:722.756
2. Nissen-Meyer, J., and I. F. Nes. 1997. Ribosomally synthesized antimicrobial peptides: their function, structure, biogenesis, and mechanism of action. Archives of Microbiology 167:67-77.
3. Riley, M. A., and D. M. Gordon. 1992. A survey of Col plasmids in natural isolates of Escherichia coli and an investigation into the stability of Col-plasmid lineages. Journal of General Microbiology 138: 1345-1352.
4. Tagg, J. R. 1992. Bacteriocins of gram-positive bacteria: an opinion regarding their nature, nomenclature, and numbers, p. 33-36. In R. 1. C. Lazdunski and F. Pattus (ed.), Bacteriocins, Microcins, and Lantabiotics. Springer-Verlag, Heidelberg, Germany.
5. Heng, N. C. K., P. A. Wescombe, J. P. Burton, R. W. Jack, and J. R. Tagg. 2007. The diversity of bacteriocins in gram-positive bacteria, p. 45-92. In M, A. Riley and M. A. Chavan (cd.), Bacteriocins: ecology and evolution. SpringerVerlag, Heidelberg, Germany.
6. Castellano, P., and G. Vignola. 2006. Inhibition of Listeria innocua and Brochothrix thermosphacta in vacuum-packaged meat by addition of bacteriocinogenic Lactobacillus curvatus CRL705 and its bacteriocins. Letters in Applied Microbiology 43: 194-199.
7. Delves-Broughton, J., P. Blackburn, R. J. Evans, and .1. Hugenholtz. 1996. Applications of the bacteriocin, nisin. Antonie Van Leeuwenhoek 69: 193-202.
8. Guinane, C. M., P. D. Cotter, C. Hill, and R. P. Ross. 2005. Microbial solutions to microbial problems; lactococcal bacteriocins for the control of undesirable biota in food. Journal of Applied Microbiology 98: 1316-1325.
9. Thomas, L. V., R. E. Ingram, H. E. Bevis, E. A. Davies, C. F. Milne, and J. Delves-Broughton. 2002. Effective use of nisin to control Bacillus and Clostridium spoilage of a pasteurized mashed potato product. Journal of Food Protection 65: 1580-1585.
10. DeKwaadsteniet, M., S. D. Todorov, H. Knoetze, and L. M. T. Dicks. 2005. Characterization of a 3944 Da bacteriocin, produced by Enterococcus mundtii ST15, with activity against gram-positive and gram-negative bacteria. International Journal of Food Microbiology 105:433-444.
11. Stern, N. J., E. A. Svetoch, B. V. Eruslanov, Y. N. Kovalev, L. I. Volodina, V. V. Perelygin, E. V. Mitsevich, I. P. Mitsevich, and V. P. Levchuk. 2005. Paenibacillus polymyxa purified bacteriocin to control Campylobacter jejuni in chickens. Journal of Food Protection 68: 1450-1453.
12. Akesson, M., M. Dufour, G. L. Sloan, and R. S. Simmonds. 2007. Targeting of streptococci by zoocin A. FEMS Microbiology Letters 270: 155-161.
13. Riley, M. A., and J. E. Wertz. 2002. Bacteriocins: evolution, ecology, and application. Annual Review of Microbiology 56: 117-137.
14. Shah, A., J. Mond, and S. Walsh. 2004. Lysostaphin-coated catheters eradicate Staphylococcus aureus challenge and block surface colonization. Antimicrobial Agents and Chemotherapy 48:2704-2707.
15. Tagg, J. R. 2004. Prevention of streptococcal pharyngitis by anti-Streptococcus pyogenes bacteriocin-like inhibitory substances (BLIS) produced by Streptococcus salivarius. Indian Journal of Medical Research 119 Suppl: 13-16.
16. Tagg, J. R., and K. P. Dierksen. 2003. Bacterial replacement therapy: adapting ‘germ warfare’ to infection prevention. Trends in Biotechnology 21:217-223.
17. Wu, J. A., C. Kusuma, J. J. Mond, and J. F. Kokai-Kun. 2003. Lysostaphin disrupts Staphylococcus aureus and Staphylococcus epidermidis biofilms on artificial surfaces. Antimicrobial Agents and Chemotherapy 47:3407-3414.
18. Oldham, E. R., and M. J. Daley. 1991. Lysostaphin: use of a recombinant bactericidal enzyme as a mastitis therapeutic. Journal of Dairy Science 74:4175-4182.
19. Twomey, D. P., A. I. Wheelock, J. Flynn, W. J. Meaney, C. Hill, and R. P. Ross. 2000. Protection against Staphylococcus aureus mastitis in dairy cows using a bismuth-based teat seal containing the bacteriocin, lacticin 3147. Journal of Dairy Science 83: 1981-1988.
20. Hert, A. P., P. D. Roberts, M. T. Momol, G. V. Minsavage, S. M. Tudor-Nelson, and J. B. Jones. 2005. Relative importance of bacteriocin-like genes in antagonism of Xanthomonas perforans tomato race 3 to Xanthomonas euvesicatoria tomato race 1 strains. Applied and Environmental Microbiology 71:3581-3588.
21. Layermicocca, P., S. L. Lonigro, F. Valerio, A. Evidente, and A. Visconti. 2002. Reduction of olive knot disease by a bacteriocin from Pseudomonas syringae pv. ciccaronei. Applied and Environmental Microbiology 68: 1403-1407.
22. Heng, N. C., N. L. Ragland, P. M. Swe, H. J. Baird, M. A. Inglis, J. R. Tagg, and R. W. Jack. 2006. Dysgalacticin: a novel, plasmid-encoded antimicrobial protein (bacteriocin) produced by Streptococcus dysgalactiae subsp. equisimilis. Microbiology 152: 1991-2001.
23. Hickey, R. M., D. P. Twomey, R. P. Ross, and C. Hill. 2003. Production of enterolysin A by a raw milk enterococcal isolate exhibiting multiple virulence factors. Microbiology 149:655-664.
24. Joerger, M. C., and T. R. Klaenhammer. 1986. Characterization and purification of helveticin J and evidence for a chromosomally determined bacteriocin produced by Lactobacillus helveticus 481. Journal of Bacteriology 167:439-446.
25. Nigutova, K., M. Morovsky, P. Pristas, R. M. Teather, H. Halo, and P. Javorsky. 2007. Production of enterolysin A by rumen Enterococcus faecalis strain and occurrence of en1A homologues among ruminal Gram-positive cocci. Journal of Applied Microbiology 102:563-569.
26. Valdes-Stauber, N., and S. Scherer. 1994. Isolation and characterization of linocin M18, a bacteriocin produced by Brevibacterium linens. Applied and Environmental Microbiology 60:3809-3814.
27. Mazzotta, A. S., Montville, T. J. 1997. Nisin induces changes in membrane fatty acid composition of Listeria monocytogenes nisin-resistant strains at 10° C. and 30° C. Journal of Applied Microbiology 82, 32-38.
28. Ainsa, J. A., N. J. Ryding, N. Hartley, K. C. Findlay, C. J. Bruton, and K. F. Chater. 2000. WhiA, a protein of unknown function conserved among gram-positive bacteria, is essential for sporulation in Streptomyces coelicolor A3(2). Journal of Bacteriology 182:5470-5478.
29. Chater, K. F. 1993. Genetics of differentiation in Streptomyces. Annual Review of Microbiology 47:685-713.
30. Chater, K. F. 1972. A morphological and genetic mapping study of white colony mutants of Streptomyces coelicolor. Journal of General Microbiology 72:9-28.
31. Gehring, A. M., J. R. Nodwell, S. M. Beverley, and R. Losick. 2000. Genomewide insertional mutagenesis in Streptomyces coelicolor reveals additional genes involved in morphological differentiation. Proceedings of the National Academy of Sciences USA 97:9642-9647.
32. Hopwood, D. A., H. Wildermuth, and H. M. Palmer. 1970. Mutants of Streptomyces coelicolor defective in sporulation. Journal of General Microbiology 61:397-408.
33. Hobbs, G., C. M. Frazer, D. C. J. Gardner, J. A. Cullum, and S. G. Oliver. 1989. Dispersed growth of Streptomyces in liquid culture. Applied Microbiology and Biotechnology 31:272-277.
34. Zhou, R., S. Chen, and P. Recsei. 1988. A dye release assay for determination of lysostaphin activity. Analytical Biochemistry 171:141-144.
35. Risoen, P. A., P. Ronning, I. K. Hegna, and A.-B. Kolsto. 2004. Characterization of a broad range antimicrobial substance from Bacillus cereus. Journal of Applied Microbiology 96:648-655
36. Laemmli, U. K. 1970. Cleavage of structural proteins during the assembly of the head of bacteriophage T4. Nature 227:680-685.
37. Sambrook, J., and D. W. Russell. 2001. Molecular Cloning: A Laboratory Manual, 3rd ed, Cold Springs Harbor Laboratory Press, Cold Springs Harbor. NY.
38. Neumann, V. C., H. E. Heath, P. A. LeBlanc, and G. L. Sloan. 1993. Extracellular proteolytic activation of bacteriolytic peptidoglycan hydrolases of Staphylococcus simulans biovar staphylolyticus. FEMS Microbiology Letters 110:205-212.
39. Kamoun, F., H. Mejdoub, H. Aouissaoui, J. Reinbolt, A. Hammami, and S. Jaoua. 2005. Purification, amino acid sequence and characterization of Bacthuricin F4, a new bacteriocin produced by Bacillus thuringiensis. Journal of Applied Microbiology 98:881-888.
40. Hopwood, D. A. 1960. Phase-contrast observations on Streptomyces coelicolor. Journal of General Microbiology 22:295-302.
41. Shirting, E. B., and D. Gottlieb. 1966. Methods for characterization of Streptomyces species. International Journal of Systematic Bacteriology 16:313-340.
42. Williams, S. T., M. Goodfellow, G. Alderson, E. M. Wellington, P. H. Sneath, and M. J. Sackin. 1983. Numerical classification of Streptomyces and related genera. Journal of General Microbiology 129:1743-1813.
43. Kampfer, P., R. M. Kroppenstedt, and W. Dott. 1991. A numerical classification of the genera Streptomyces and Streptoverticillium using miniaturized physiological tests. Journal of General Microbiology 137:1831-1891.
44. Olson, J. B., D. K. Harmody, and P. J. McCarthy. 2002. Alpha-proteobacteria cultivated from marine sponges display branching rod morphology. FEMS Microbiology Letters 211:169-173.
45. Farris, M. H., and J. B. Olson. 2007. Detection of actinobacteria cultivated from environmental samples reveals bias in universal primers. Letters in Applied Microbiology 45:376-81.
46. Hall, T. A. 1999. BioEdit: a user-friendly biological sequence alignment editor and analysis program for Windows 95/98/NT. Nucleic Acids Symposium Series 41:95-98.
47. Thompson, J. D., D. G. Higgins, and T. J. Gibson. 1994. CLUSTAL W: improving the sensitivity of progressive multiple sequence alignment through sequence weighting, position-specific gap penalties and weight matrix choice. Nucleic Acids Research 22:4673-4680.
48. Saitou, N., and M. Nei. 1987. The neighbor-joining method: a new method for reconstructing phylogenetic trees. Molecular Biology and Evolution 4:406-425.
49. Swofford, D. L. 2002. PAUP*. Phylogenetic analysis using parsimony (*and other methods), 4.0 ed. Sinauer Associates, Sunderland, Mass.
50. Kimura, M. 1980. A simple method for estimating evolutionary rates of base substitutions through comparative studies of nucleotide sequences. Journal of Molecular Evolution 16:111-120.
51. Felsenstein, J. 1981. Evolutionary trees from DNA sequences: a maximum likelihood approach. Journal of Molecular Evolution 17:368-376.
52. Hatano, K., T. Nishii, and H. Kasai. 2003. Taxonomic re-evaluation of whorl-forming Streptomyces (formerly Streptoverticillium) species by using phenotypes, DNA-DNA hybridization and sequence of gyrB, and proposal of Streptomyces luteireticuli (ex Katoh and Arai 1957) corrig., sp. nov., nom. rev. International Journal of Systematic and Evolutionary Microbiology 53:1519-1529.
53. Williams, S. T., M. Goodfellow, G. Alderson, E. M. Wellington, P. H. Sneath, and M. J. Sackin. 1983. Numerical classification of Streptomyces and related genera. Journal of General Microbiology 129:1743-1813.
54. Kampfer, P., R. M. Kroppenstedt, and W. Dott. 1991. A numerical classification of the genera Streptomyces and Streptoverticillium using miniaturized physiological tests. Journal of General Microbiology 137:1831-1891.
55. Buchanan, R. E., and N. E. Gibbons (ed.). 1974. Bergey's Manual of Determinative Bacteriology, 8th ed. The Williams & Wilkins Co., Baltimore, Md.

TABLE 1
Comparison of morphological, cultural, and physiological
characteristics of strain RB72 and related Streptomyces species.

| Character | 1 | 2 | 3 | 4 | 5 | 6 |
|---|---|---|---|---|---|---|
| Morphology and | | | | | | |
| pigmentation: | | | | | | |
| Aerial mass on | white | beige | violet | gray | red-gray | gray |
| oatmeal agar | | | | | | |
| Spore-chain | − | | SP | SP | RF | SP |
| arrangement | | | | | | |
| Spore surface | − | | spiny | smooth | smooth | smooth |
| Melanin production | − | − | + | + | + | + |
| Production of | − | | | − | − | + |
| diffusible pigments | | | | | | |
| Hydrolysis of: | | | | | | |
| Adenine | + | − | | + | + | + |
| Casein | + | + | | + | + | + |
| Cellulose | − | | | | | |
| Esculin | + | − | | | | |
| Gelatin | + | | | + | + | + |
| Hippurate | | | | − | | + |
| Hypoxanthine | + | | | + | + | + |
| L-Tyrosine | + | | | + | + | + |
| Starch | + | + | | + | + | + |
| Xanthine | + | + | | + | + | + |
| Growth on sole carbon | | | | | | |
| sources (1%, w/v): | | | | | | |
| No Carbon | − | − | − | − | − | − |
| D-Glucose | + | + | + | + | + | + |
| L-Arabinose | + | − | + | + | + | − |
| D-Fructose | − | d | − | + | + | + |
| D-Galactose | + | | + | − | − | + |
| i-Inositol | + | + | − | − | − | − |
| D-Mannitol | − | − | − | − | + | + |
| D-Raffinose | + | − | + | − | d | − |
| L-Rhamnose | + | − | − | + | d | + |
| Sucrose | − | − | − | − | d | − |
| D-Xylose | + | − | − | + | + | + |
| Sorbitol | − | | | | | |
| Cellobiose | + | | | + | + | + |
| D-Melibiose | + | | | − | d | d |
| L-Sorbose | − | | | − | | |
| D-Maltose | + | | | + | | |
| Adonitol | + | | | − | − | − |
| D-Lactose | + | | | − | + | + |
| D-Mannose | + | | | + | + | + |
| Dextrin | + | | | | | |
| Inulin | − | | | − | − | + |

TABLE 2
Spectrum of bacteriocin activity of the
producer strain Streptomyces sp. strain RB72.

| Organism | Source or reference number | Inhibition |
|---|---|---|
| Actinomycetes | | |
| Streptomyces sp. strain RB72 | woodland bluff in Lynn, | − |
| (producer strain) | Alabama | |
| S. avermitilis MA-4680 | USDA^{a}; NRRL 8165 | + |
| S. coelicolor A3(2) | DSMZ^{b}; DSM 40783 | + |
| S. lividans 66 | USDA; NRRL B-12275 | + |
| S. venezuelae | USDA; NRRL ISP-5230 | + |
| Nocardia salmonicida | USDA; NRRL B-2778 | + |
| Nocardia vaccinii | USDA; NRRL WC-3500 | + |
| Rhodococcus marinonascens | DSMZ; DSM 43752 | + |
| Other Gram-positive bacteria | | |
| Bacillus megaterium | ATCC^{c}; 14581 | + |
| Bacillus subtilis 168 | ATCC; 23857 | + |
| Enterococcus faecalis | ATCC; 29212 | + |
| Micrococcus luteus | Ward's Nat. Science; | + |
| | 85W0966 | |
| Staphylococcus aureus FDA209 | ATCC; 6538 | + |
| Streptococcus pyogenes | ATCC; 49615 | + |
| Gram-negative bacteria | | |
| Escherichia coli DH10B | Invitrogen Corp.; | + |
| | Carlsbad, CA | |
| Klebsiella pneumoniae | ATCC; 13883 | + |

^{a}United States Department of Agriculture ARS Culture Collection (Peoria, IL)
^{b}DSMZ German Collection of Microorganisms and Cell Culture (Braunschweig, Germany)
^{c}American Type Culture Collection (Manassas, VA)

TABLE 3
Factors affecting the bacteriolytic activity of BLIS RB72

| | Reaction | Residual |
|---|---|---|
| Treatment | Duration/Condition | activity (%)* |
| pH | | |
| 2 | 4 h/4° C. | 0 |
| 3 | 4 h/4° C. | 6 |
| 4 | 4 h/4° C. | 65 |
| 5 | 4 h/4° C. | 70 |
| 6 | 4 h/4° C. | 87 |
| 7 | 4 h/4° C. | 100 |
| 8 | 4 h/4° C. | 100 |
| 9 | 4 h/4° C. | 100 |
| Temperature (° C.) | | |
| −20 | 30 days | 100 |
| 4 | 30 days | 100 |
| 25 | 24 h | 100 |
| 37 | 30 min | 100 |
| 40 | 30 min | 95 |
| 55 | 30 min | 43 |
| 60 | 30 min | 16 |
| 80 | 30 min | 0 |
| 100 | 30 min | 0 |
| 121 | 15 min/304 kPa | 0 |
| Enzymes | | |
| α-Chymotrypsin | 4 h/37° C./1 mg/ml | 60 |
| Proteinase K | 4 h/37° C./10 mg/ml | 11 |
| Trypsin | 4 h/37° C./1 mg/ml | 30 |

TABLE 4
Conservative amino acid substitutions.

| Amino Acid Substitutions | |
|---|---|
| Original Residue | Exemplary Substitutions |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An isolated Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria.

2. The bacterium of claim 1, wherein the bacterium is deposited under ARS Culture Collection No. NRRL B-24574^{T}, and under DSMZ general collection accession number DSM 41917.

3. The bacterium of claim 1, wherein the bacterium comprises a 16S rRNA nucleic acid sequence identified as SEQ ID NO:1, wherein the nucleic acid sequence is deposited under GenBank Accession No. EF657884.

4. The bacterium of claim 3, wherein the 16S rRNA nucleic acid sequence is greater than 98.87% identical to the nucleic acid sequence identified as SEQ ID NO:1.

5. The bacterium of claim 1, wherein the bacteriocin comprises a polypeptide comprising a 15 amino acid sequence identified as SEQ ID NO: 2.

6. The bacterium of claim 5, wherein the polypeptide has a weight of about 27 kDa as visualized by SDS-PAGE.

7. An isolated polypeptide comprising a 15 amino acid sequence identified as SEQ ID NO: 2, wherein the polypeptide has a weight of about 27 kDa as visualized by SDS-PAGE, and fragments thereof.

8. The polypeptide of claim 7, wherein the polypeptide, or one or more fragments thereof, kills or inhibits the growth of Gram-positive or Gram-negative target bacteria.

9. The polypeptide of claim 8, wherein the Gram-positive target bacteria are selected from the group consisting of Streptomyces avermitilis, Streptomyces coelicolor, Streptomyces lividans, Streptomyces venezuelae, Nocardia salmonicida, Nocardia vaccinii, Rhodococcus marinonascens, Bacillus megaterium, Bacillus subtilis, Bacillus cereus, Enterococcus faecalis, Micrococcus luteus, Staphylococcus aureus, Streptococcus sp., Streptococcus pyogenes, Listeria monocytogenes, Clostridium perfringens, Clostridium botulinum, Lactococcus cremoris, Lactobacillus sp., and Leuconostoc sp.

10. The polypeptide of claim 8, wherein the Gram-negative target bacteria are selected from the group consisting of Escherichia coli DH10B, Klebsiella pneumoniae, Salmonella typhimurium, Salmonella enteritidis, Campylobacter jejuni, Yersinia enterocolitica, Yersinia pseudotuberculosis, Vibrio cholerae O1 and non-O1, Vibrio parahaemolyticus, Vibrio vulnificus, Aeromonas hydrophila, Plesiomonas shigelloides, Shigella sonnei, Shigella flexneri, Enterobacter aerogenes, Flavobacterium sp., Acinetobacter sp., and Proteus sp.

11. The polypeptide of claim 8, wherein the polypeptide, and fragments thereof, retain activity when stored at a temperature from about −20° C. to about 60° C.

12. The polypeptide of claim 8, wherein the polypeptide, or one or more fragments thereof, kills or inhibits the growth of Gram-positive or Gram-negative target bacteria at a temperature from about 4° C. to about 60° C.

13. The polypeptide of claim 12, wherein the polypeptide, or one or more fragments thereof, kills or inhibits the growth of Gram-positive or Gram-negative target bacteria at a temperature of about 37° C.

14. The polypeptide of claim 8, wherein the polypeptide and fragments thereof retain activity when stored at a pH from about 3 to about 9.

15. The polypeptide of claim 14, wherein the polypeptide, or one or more fragments thereof, kills or inhibits the growth of Gram-positive or Gram-negative target bacteria at a pH of about 7.

16. An isolated nucleic acid comprising a nucleotide sequence identified as SEQ ID NO:3, wherein the nucleic acid encodes the polypeptide of claim 7.

17. A composition comprising the polypeptide of claim 8 and a carrier.

18. A method of killing or inhibiting the growth of a Gram-positive or Gram-negative target bacterium, comprising contacting the target bacterium with an effective amount of a bacteriocin comprising a polypeptide comprising a 15 amino acid sequence identified as SEQ ID NO: 2, wherein the polypeptide has a weight of about 27 kDa as visualized by SDS-PAGE, wherein the polypeptide, or one or more fragments thereof, kills or inhibits the growth of Gram-positive or Gram-negative target bacteria, whereby contacting the target bacterium with the bacteriocin kills or inhibits the growth of the target bacterium.

19. The method of claim 18, wherein the target bacteria are selected from the group group consisting of Streptomyces avermitilis, Streptomyces coelicolor, Streptomyces lividans, Streptomyces venezuelae, Nocardia salmonicida, Nocardia vaccinii, Rhodococcus marinonascens, Bacillus megaterium, Bacillus subtilis, Bacillus cereus, Enterococcus faecalis, Micrococcus luteus, Staphylococcus aureus, Streptococcus sp., Streptococcus pyogenes, Listeria monocytogenes, Clostridium perfringens, Clostridium botulinum, Lactococcus cremoris, Lactobacillus sp., Leuconostoc sp., Escherichia coli DH10B, Klebsiella pneumoniae, Salmonella typhimurium, Salmonella enteritidis, Campylobacter jejuni, Yersinia enterocolitica, Yersinia pseudotuberculosis, Vibrio cholerae O1 and non-O1, Vibrio parahaemolyticus, Vibrio vulnificus, Aeromonas hydrophila, Plesiomonas shigelloides, Shigella sonnei, Shigella flexneri, Enterobacter aerogenes, Flavobacterium sp., Acinetobacter sp., and Proteus sp.

20. A method of growing a Gram-positive non-motile, non-spore-forming, aerobic bacterium designated Streptomyces scopuloiridis, wherein the bacterium produces a bacteriocin that kills or inhibits the growth of Gram-positive or Gram-negative target bacteria, comprising culturing the bacterium at a temperature and in a medium effective to promote growth of the bacterium.

21. The method of claim 20, wherein the bacterium is grown on mannitol soya flour agar, nutrient agar with 0.4% dextrose, yeast extract-malt extract agar (International Streptomyces Project medium 2), or oatmeal agar (International Streptomyces Project medium 3) at a temperature of about 25° C.

22. A method of preserving food or preventing bacterial contamination of food, comprising contacting the food with an effective amount of a bacteriocin comprising a polypeptide comprising a 15 amino acid sequence identified as SEQ ID NO: 2, wherein the polypeptide has a weight of about 27 kDa as visualized by SDS-PAGE, wherein the polypeptide, or one or more fragments thereof, kills or inhibits the growth of Gram-positive or Gram-negative target bacteria, whereby contacting the food with the bacteriocin preserves the food or prevents bacterial contamination of the food.

23. A method of preventing bacterial contamination of a surface of an object, comprising contacting the surface of the object with an effective amount of a bacteriocin comprising the polypeptide of claim 8, whereby contacting the surface of the object with the bacteriocin prevents bacterial contamination of the surface of the object.
